(19) United States
(12) Patent Application Publication
(10) Pub. No.: US 2007/0270755 A1
(43) Pub. Date: Nov. 22, 2007

(54) GUIDEWIRE HANDLING DEVICE

(75) Inventors: Randolf Von Oepen, Los Altos Hills, CA (US); Richard Newhauser, San Francisco, CA (US); Travis Yribarren, San Mateo, CA (US)

Correspondence Address: SQUIRE, SANDERS & DEMPSEY L.L.P., 1 MARITIME PLAZA, SUITE 300, SAN FRANCISCO, CA, 94111, US

(73) Assignee: Abbott Laboratories, Abbott Park, IL (US)

(21) Appl. No.: 11/738,420

(22) Filed: Apr. 20, 2007

Related U.S. Application Data
(60) Provisional application No. 60/793,783, filed on Apr. 21, 2006.

(51) Int. Cl. A61M 25/09

(52) U.S. Cl. 604/164.13; 600/585

(57) ABSTRACT

This invention describes a guidewire handling device for creating an indicium and/or urging a guidewire. A guidewire handling device is provided that includes a device body having a guidewire passage sized to receive a guidewire. A clamping member is coupled to the device body and is movable between a first and second position. The guidewire handling device includes a visible marker that provides visible feedback regarding the angular orientation of the guidewire handling device. In addition, the guidewire handling device includes a contoured surface that allows the torque applied to the guidewire handling device to be dependent on the location where the torque is applied.

This application claims the benefit of U.S. Provisional Application No. 60/793,783, filed Apr. 21, 2006, the entire disclosure of which is incorporated herein by reference.

### BACKGROUND OF THE INVENTION

1. Field of the Invention

This invention generally relates to devices for handling thin wires and cables. More particularly, it relates to a device that can be used to handle a guidewire in any of a variety of medical procedures used to treat medical conditions in a human or animal patient.

2. Description of Related Art

Interventional medical procedures using a guidewire are well known. These procedures generally include the use of a guidewire to access body vessels such as coronary vessels. Access is achieved by inserting and tracking a guidewire through the body vessel system to the desired location. Rotating and traversing the guidewire controls this tracking.

It can be difficult to manipulate a guidewire manually. Since guidewires generally have small diameters and low friction coatings, they are challenging to grip. Therefore, the use of a secondary handling device to grip and impart motion to the guidewire is commonly used by interventional physicians.

Although secondary handling devices, also commonly referred to as torque devices or torquers, improve guidewire control, they also have shortcomings. These deficiencies include designs that make loading over a guidewire cumbersome, have no active feedback indicating angular displacement of the torquer, and include geometries that do not facilitate comfortable use or effective torque control. This invention provides a guidewire handling device to address these shortcomings, and to provide additional benefits that enhance the user experience.

### SUMMARY OF THE INVENTION

The purpose and advantages of the present invention will be described and apparent from the description that follows, and through the practice of the invention.

To achieve these purposes and advantages, and in accordance with the present invention, a guidewire handling device is provided that includes a device body having a guidewire passage sized to receive a guidewire. A clamping member is coupled to the device body and is movable between a first and second position. The guidewire handling device includes a visible marker that provides visible feedback regarding the angular orientation of the guidewire handling device. In addition, the guidewire handling device includes a contoured surface that allows the torque applied to the guidewire handling device to be dependent on the location where the torque is applied.

In further accordance with the present invention, the guidewire passage may be a groove, hole, or combination thereof, that is disposed within the device body.

In further accordance with the present invention, the clamping member may be a lever mechanism that is pivotably associated with the device body. In a first clamping member position, an axis of the guidewire passage is not obstructed and a guidewire is insertable therein.

In a further aspect of the present invention, the visible marker can be defined by an edge of the guidewire handling device. For example, the edge of the device body adjacent to the clamping member and substantially aligned with the guidewire passage can function as a visible marker.

In further accordance with the present invention, there is a wire marking feature associated with the clamping member. The wire marking feature is capable of creating an indicium on the guidewire, which allows a physician to differentiate the guidewire from another guidewire.

In an alternative embodiment of the present invention, the clamping member can be slidably associated with the device body.

In an alternative embodiment of the present invention, the visible marker can also be a boss, print, or a combination thereof.

In an alternative embodiment of the present invention, the wire marking feature can include a transferable ink, an abrasive surface, a cutting surface or a combination thereof.

In accordance with the present invention, a method can be provided for urging a guidewire that includes receiving a guidewire within the guidewire passage of the guidewire handling device, actuating the clamping member between a first and second position, sandwiching the guidewire between the device body and the clamping member, and applying a force to a surface of the guidewire handling device.

In further accordance with the present invention, a method can be provided for creating an indicium on a guidewire that includes receiving a guidewire within a guidewire passage of a guidewire handling device and actuating a clamping member coupled to the device body, thereby sandwiching the guidewire between a wire marking feature and the device body and creating a noticeable guidewire deformation. Optionally, the guidewire handling device may then be moved relative to the guidewire, thereby skiving a surface of the guidewire.

### BRIEF DESCRIPTION OF THE DRAWINGS

For a further appreciation of the above and other advantages, reference is made to the following detailed description and to the drawings, in which:

FIG. 1 is a perspective view of an exemplary embodiment of the guidewire handling device in accordance with the present invention illustrating the clamping member in a first position and a guidewire received within the guidewire passage;

FIG. 2 is a plan view of the exemplary embodiment of the guidewire handling device according to the present invention illustrating the clamping member in a first position and a guidewire received within the guidewire passage;

FIG. 3 is a perspective view of the exemplary embodiment of the guidewire handling device according to the present invention illustrating the clamping member in a second position and a guidewire received within the guidewire passage;

FIG. 4 is a plan view of a second end of the exemplary embodiment of the guidewire handling device in accordance with the present invention illustrating the clamping member in a second position;

FIG. 5 is a cross-sectional view in accordance with the present invention taken about line A-A of FIG. 4 illustrating the guidewire sandwiched between the marking feature and the clamping insert;

FIG. 6 is a perspective view of an alternative embodiment of the guidewire handling device having a clamping member slidably coupled to the device body according to the present invention;

FIG. 7 is a plan view of the alternative embodiment of the guidewire handling device;

FIG. 8 is a cross-sectional view of the alternative embodiment of the present invention taken about line B-B of FIG. 7 illustrating the clamping member in a first position; and

FIG. 9 is a cross-sectional view of the alternative embodiment of the present invention taken about line B-B of FIG. 7 illustrating the clamping member in a second position.

### DETAILED DESCRIPTION OF THE PREFERRED EMBODIMENTS

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various accompanying figures.

In accordance with the present invention, there is provided a guidewire handling device for facilitating the handling of a guidewire during an interventional procedure. The device is particularly useful for transmitting loads to the guidewire thereby causing it to rotate and traverse through a vascular system to a desired treatment location. The device includes other features and operations that provide advantages that will be clear from the following detailed description.

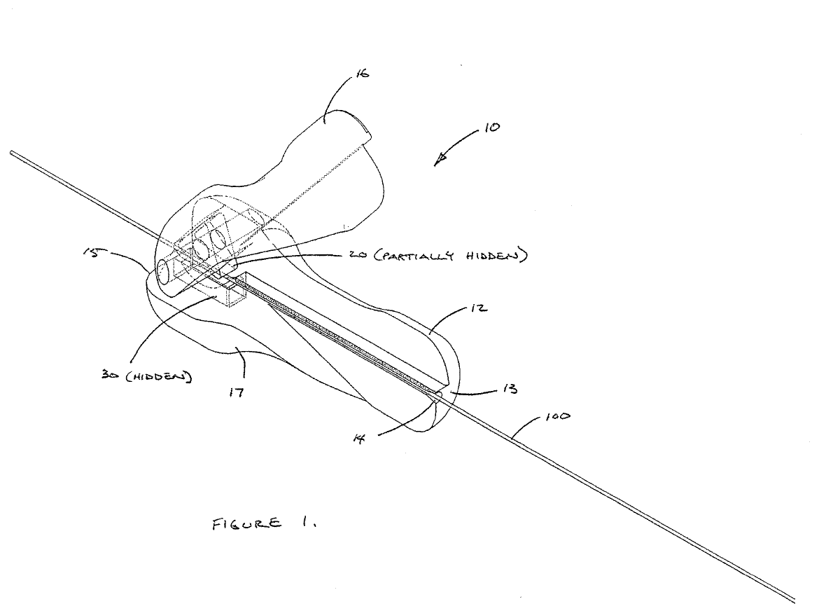

Referring now to FIG. 1, there is shown an exemplary embodiment of the guidewire handling device 10 in accordance with the present invention. The guidewire handling device 10 has a device body 12 and a clamping member 16 that are pivotably coupled. There is a wire marking feature 20 associated with and movable in conjunction with the clamping member 16. The device body 12 has a clamping insert 30 associated therewith. The clamping insert is optional given that the device body could be designed to act as a clamping surface. The guidewire handling device 10 further includes a visible marker 200.

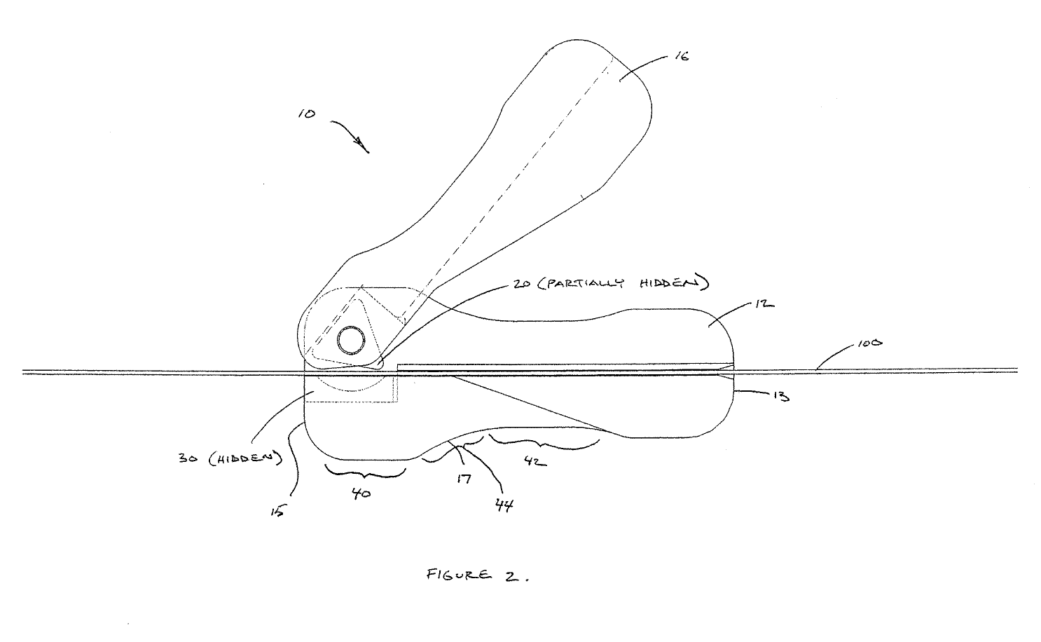

The device body 12 includes a first end 13 and a second end 15. The device body 12 further includes a surface 17 therebetween. The surface 17 is preferably contoured to improve the handling. Referring now to FIG. 2, the contoured surface 17 is shown to have a large profile region 40 and a small profile region 42 with a generally tapered region 44 therebetween.

Referring again to FIG. 1, the device body 12 is shown to include a guidewire passage 14. The guidewire passage 14 is sized to receive a guidewire 100. In a further aspect of this embodiment, the guidewire passage 14 is a groove with a first end adjacent the first end of the device body 12.

It an alternative embodiment of the present invention, the guidewire passage 14 further includes a hole.

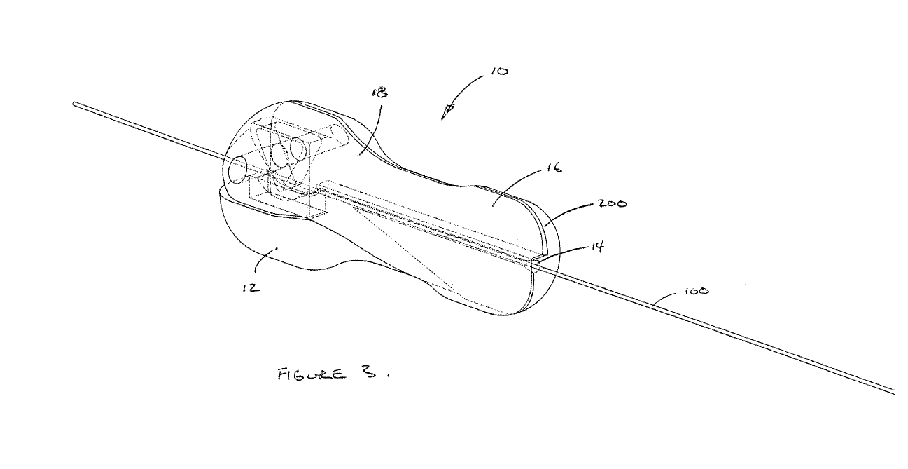

Referring now to FIG. 3, the clamping member 16 is shown to have a contoured surface 18. The clamping member 16 is movable between a first and second position. In the first position, the axis of the guidewire passage 14 is unobstructed and a guidewire may be received within the guidewire passage 14 between the clamping member 16 and the device body 12. In a second position, the contoured surface 18 of the clamping member 16 is complementary to the contoured surface 17 of the device body 12, thereby providing a configuration for the guidewire handling device 10 that is comfortable to grip and manipulate.

Referring now to FIG. 2, there is shown a wire marking feature 20 that is partially hidden by the clamping member 16. In this exemplary embodiment, the wire marking feature 20 is fixedly associated with the clamping member 16 and thereby movable in conjunction with the clamping member 16. The wire marking feature 20 is shown to be fulcrum shaped. It is also possible to integrally form the clamping member and the marking feature.

As shown in FIG. 2, there is a clamping insert 30 in accordance with the present invention, hidden by the device body 12 and fixedly associated with the device body 12. The clamping insert 30 has an arcuate surface shaped similar to the path transcribed by the wire marking feature 20 when the clamping member 16 is moved from a first position to a second position.

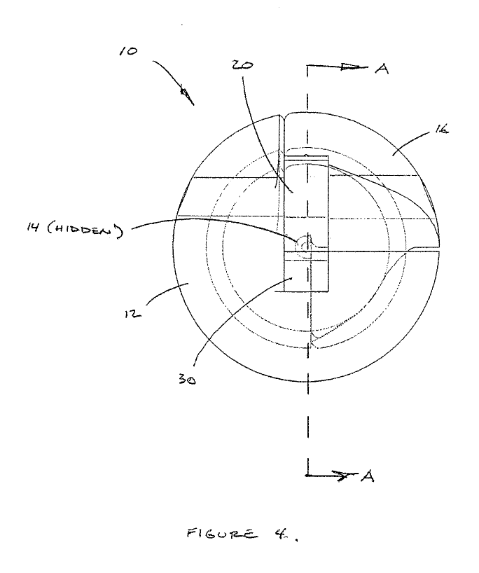

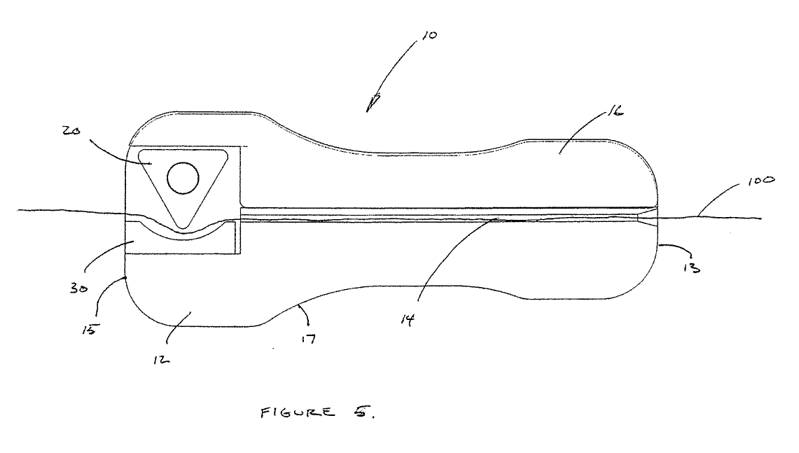

Referring now to FIG. 4, there is shown an end view of the guidewire handling device 10 with the clamping member 16 in a second position. In this configuration, the wire marking feature 20 obstructs the guidewire passage 14. Referring to FIG. 5, there is shown a cross-sectional view of the guidewire handling device 10 in accordance with the present invention taken about line A-A of FIG. 4. The guidewire is permanently deformed by, and sandwiched between, the wire marking feature 20 and the clamping insert 30 when the clamping member 16 is in a second position.

Referring now to FIG. 3, there is shown a visible marker 200 defined by an edge of the device body 12. This edge is preferably made visible by the contrast in surface characteristics of the device body 12 compared to the clamping member 16. The visible marker 200 is substantially aligned with the axis of the guidewire passage 14.

It is further contemplated that the wire marking feature 20 and clamping member 16 can be integrally formed within the clamping member 16 and device body 12, respectively.

In an alternative embodiment of the present invention, it is contemplated that the wire marking feature 20 and clamping member 16 may be designed so as to not permanently deform the guidewire when the clamping member 16 is actuated to a second position. In a further aspect of this embodiment, the marking feature may include transferable ink that is transferred to the guidewire surface, thereby providing a visible indicium.

In an alternative embodiment of the present invention, the marking feature can include an abrasive surface. The abrasive surface can contact the guidewire surface when the clamping member 16 is actuated to a second position.

It is further contemplated that the visible marker 200 in accordance with the present invention may be a boss, print, or combination thereof, located on the surface of the guidewire handling device 10.

The device body 12 and clamping member 16 may be injection molded from a thermoplastic material such as ABS or Delrin. It is contemplated that other materials may be used such as other plastics, filled plastics, ceramics, and metals. It is further contemplated that the device body 12 and clamping member 16 may be formed using other techniques such as conventional machining methods.

The wire marking feature 20 and clamping insert 30 are preferably constructed from a material that is at least as hard as the material used to construct the device body 12 and clamping member 16. Preferably, the wire marking feature 20 and clamping insert 30 are formed using conventional machining methods from a metal such as carbide or hardened steel. It is contemplated that other materials may be used such as plastics, filled plastics, and ceramics. It is further contemplated that the wire marking feature 20 and clamping insert 30 may be formed using other techniques such as metal casting.

In accordance with the present invention, a method is provided for urging a guidewire using the guidewire handling device 10. A guidewire is first received within the guidewire passage 14 when the clamping member 16 is in a first position. This is achieved by inserting the guidewire between the clamping member 16 and the device body 12. The clamping member 16 is actuated between a first and second position, thereby causing the wire marking feature 20 to pivot and sandwich the guidewire against the clamping insert 30. The operator may then apply a force to the guidewire handling device 10 to transmit the load to the guidewire, thereby urging it to respond as desired.

In further accordance with the present invention, a method is provided for using the guidewire handling device 10 to create an indicium in the guidewire. A guidewire is first received within the guidewire passage 14 when the clamping member 16 is in a first position. The guidewire is inserted between the clamping member 16 and the device body 12. The clamping member 16 is actuated, thereby bringing the wire marking feature 20 into contact with the guidewire, and deforming the guidewire as the clamping member 16 reaches a second position. The deformation of the guidewire acts as an indicium on the guidewire after it is removed from the guidewire handling device 10.

In an alternative embodiment of the present invention, a method is provided for using the guidewire handling device 10 to create a visible indicium in the guidewire. A guidewire is first received within the guidewire passage 14 when the clamping member 16 is in a first position. The guidewire is inserted between the clamping member 16 and the device body 12. The clamping member 16 is actuated, thereby bringing the wire marking feature 20 into contact with the guidewire, and transferring an ink to the surface of the guidewire as the clamping member 16 reaches a second position. The ink marked surface of the guidewire acts as an indicium on the guidewire after it is removed from the guidewire handling device 10.

In an alternative embodiment of the present invention, a method is provided for using the guidewire handling device 10 to create a visible indicium in the guidewire. A guidewire is first received within the guidewire passage 14 when the clamping member 16 is in a first position. The guidewire is inserted between the clamping member 16 and the device body 12. The clamping member 16 is actuated, thereby bringing the wire marking feature 20 having an abrasive surface into contact with the guidewire as the clamping member 16 reaches a second position. The guidewire handling device 10 is then moved relative to the guidewire, thereby causing a surface of the guidewire to be skived by the abrasive surface of the marking feature. The skived surface of the guidewire acts as an indicium on the guidewire after it is removed from the guidewire handling device 10.

In addition to providing a device that is capable of handling and transmitting forces to the guidewire, this invention has several other advantages. The guidewire handling device 10 can be loaded onto the guidewire at any location without the need for advancing the device axially over the guidewire. This advantageous feature makes the guidewire handling device 10 suitable for use with guidewires of any length. Therefore, the device is especially suitable for use during interventional procedures with longer guidewires, such as during the treatment of chronic total occlusions. Typical guidewire lengths are 180 cm or 300 cm, however this device will work with guidewires of all sizes.

The guidewire handling device 10 advantageously provides a contoured surface that is comfortable to grip. In addition, since the guidewire handling device 10 can be gripped in locations with larger or smaller profiles, the torque transmission can be varied according to the grip location. This provides the physician with better control of the guidewire tip while traversing the guidewire to the desired location within the vessel system. Additionally, this design is ergonomically suitable for a variety of hand sizes.

In a further aspect of the present invention, the visible marker 200 advantageously provides visual feedback to the physician regarding the angular position and movement of the torque device. This feedback provides the physician with better control of the guidewire tip while traversing the guidewire to the desired location with the vessel system.

In accordance with a further aspect of the present invention, the guidewire handling device 10 also advantageously creates an indicium in the guidewire, thereby making the guidewire distinguishable. This is of particular importance when multiple guidewires are used during an interventional procedure. For example, during the treatment of a bifurcated vessel, a guidewire is commonly positioned in the main branch vessel and a guidewire is commonly positioned in the side branch vessel. These guidewires must be distinguished from each other, but this is difficult since the guidewires can appear identically at their proximal ends. By using a guidewire handling device 10 with a wire marking feature 20 on one of the guidewires, an indicium is created that allows the physician to identify the guidewire, thereby distinguishing it from other guidewires.

In a further aspect of the present invention, the guidewire handling device 10 is advantageously suitable for use with a variety of guidewire diameters. The clamping mechanism and guidewire passage 14 can be sized to receive and sandwich guidewires of any size.

### Alternative Embodiment

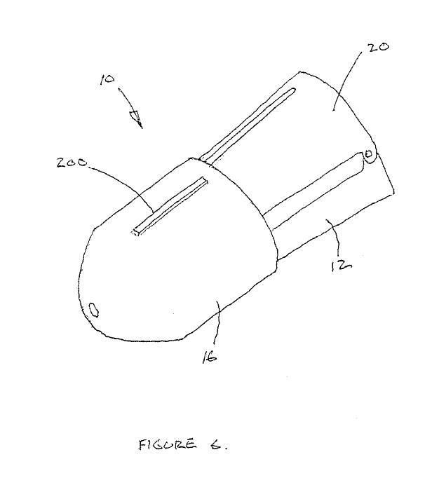

Referring now to FIG. 6, there is shown an alternative embodiment of the guidewire handling device 10 in accordance with the present invention. The guidewire handling device 10 has a device body 12 and a clamping member 16 that are slidably coupled. There is a wire marking feature 20 associated with and movable in conjunction with the clamping member 16. The device body 12 has a clamping insert 30 (not shown) associated therewith. The guidewire handling device 10 further includes a visible marker 200.

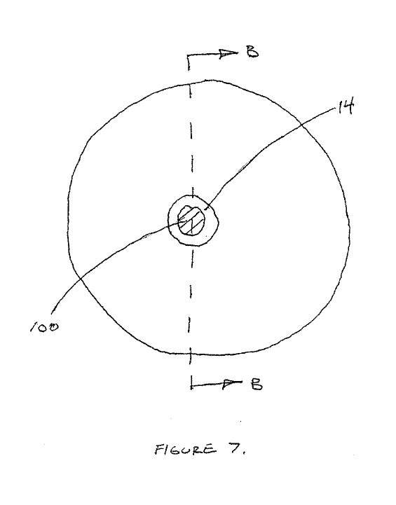

Referring again to FIG. 7, an end view of the guidewire handling device is shown in accordance with the present invention, a guidewire passage 14 is shown placed therethrough. The guidewire passage 14 is sized to receive a guidewire 100.

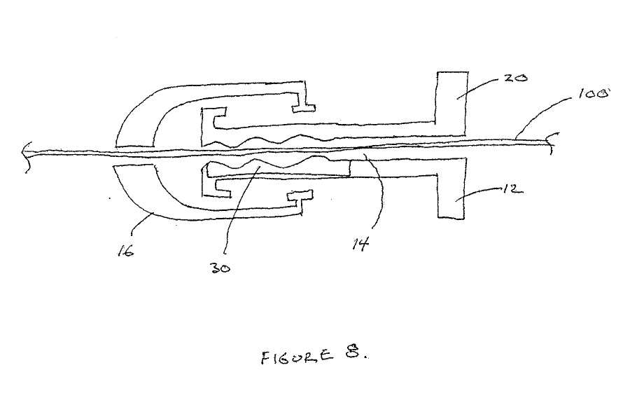

Referring now to FIG. 8, there is shown a cross-sectional view in accordance with the present invention taken about line B-B of FIG. 7. The clamping member 16 is movable between a first and second position. In the first position illustrated in FIG. 8, the axis of the guidewire passage 14 is unobstructed and a guidewire 100 may be received within the guidewire passage 14 between the clamping member 16 and the device body 12.

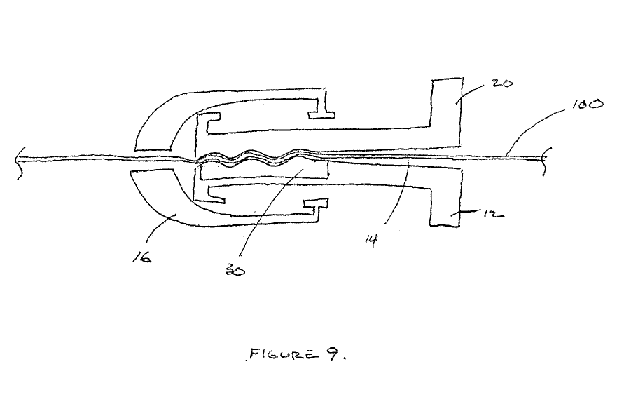

Referring now to FIG. 9, there is shown a cross-sectional view in accordance with the present invention taken about line B-B of FIG. 7. The clamping member 16 is shown in a second position. The clamping member 16 acts upon the device body 12, thereby sandwiching the guidewire between the wire marking feature 20 and the clamping insert 30.

Referring again to FIG. 6, there is shown a visible marker 200 positioned on the guidewire handling device 10. In this embodiment, the visible marker 200 is a boss placed on the surface of the clamping member 16. As discussed previously, the visible marker may be formed in other ways such as:

1. Machining

2. Pad Printing

3. Laser Printing

4. Ink Transfer

While the preferred embodiments of the invention have been illustrated and described, it will be appreciated that various changes can be made without departing from the scope of the invention. It is therefore intended that the scope of the invention be determined from the following claims and equivalents thereof.

What is claimed is:

1. A guidewire handling device, comprising:
(a) a device body having a guidewire passage for receiving a guidewire;
(b) a clamping member coupled to the device body, said clamping member being movable between a first position and a second position;
(c) a visible marker disposed thereon, whereby angular orientation of a guidewire handling device is discernible; and
(d) a surface whereby a torque applied to the guidewire handling device is dependent upon the location in which the torque is applied.

2. The device of claim 1, wherein the guidewire passage is a groove disposed upon the device body.

3. The device of claim 1, wherein the guidewire passage is a hole disposed through the device body.

4. The device of claim 1, wherein the clamping member is pivotably coupled to the device body.

5. The device of claim 1, wherein the clamping member is slidably coupled to the device body.

6. The device of claim 1, wherein the visible marker is defined by an edge of the guidewire handling device.

7. The device of claim 1, wherein the visible marker is a boss.

8. The device of claim 1, wherein the visible marker is a print.

9. The device of claim 1, further comprising a wire marking feature associated with the clamping member, whereby the wire marking feature creates an indicium upon the guidewire.

10. The device of claim 9, wherein the wire marking feature is fulcrum shaped.

11. The device of claim 9, wherein the wire marking feature further includes transferable ink.

12. The device of claim 9, wherein the wire marking feature further includes an abrasive surface.

13. A method of urging a guidewire using a guidewire handling device, comprising the steps of:
(a) receiving a guidewire within a guidewire passage of a device body;
(b) actuating a clamping member coupled to the device body, thereby sandwiching the guidewire between the clamping member and the device body; and
(c) applying a force to a surface of the guidewire handling device, whereby force is transmitted to the guidewire.

14. A method of creating an indicium on a guidewire using a guidewire handling device, comprising the steps of:
(a) receiving a guidewire within a guidewire passage of a device body; and
(b) actuating a clamping member coupled to the device body, thereby sandwiching the guidewire between a wire marking feature and the device body.

15. The method of claim 14, further comprising the step of moving the guidewire handling device relative to the guidewire, thereby skiving a surface of the guidewire.
